(19) United States
(12) Patent Application Publication
(10) Pub. No.: US 2011/0301475 A1
(43) Pub. Date: Dec. 8, 2011

(54) VOLTAGE-FREQUENCY CONVERSION CIRCUIT AND BLOOD PRESSURE MEASUREMENT DEVICE EQUIPPED WITH SAME

(75) Inventor: Eisuke Yamazaki, Takarazuka-shi (JP)

(73) Assignee: OMRON HEALTHCARE Co., Ltd., Kyoto-shi (JP)

(21) Appl. No.: 13/214,584

(22) Filed: Aug. 22, 2011

Related U.S. Application Data
(63) Continuation of application No. PCT/JP2010/051877, filed on Feb. 9, 2010.

(30) Foreign Application Priority Data
Feb. 26, 2009 (JP) 2009-043953

(51) Int. Cl. A61B 5/022; H03D 3/00

(52) U.S. Cl. 600/493; 327/102

(57) ABSTRACT

A resistor element is arranged between an input terminal and a node. A switch element is arranged between the node and a ground voltage, and is conducted according to a voltage level of the node. A resistor element is arranged between the nodes. A resistor element is arranged between the node and one side of the input node of an NOR circuit. A capacitor is connected between the nodes. The input node of the NOR circuit is connected to the node through the resistor element and a ground voltage. The input node of an NOR circuit is connected to an output node of the NOR circuit and the ground voltage. The input node of the NOR circuit is connected to the node and the ground voltage.

### TECHNICAL FIELD

The present invention relates to a voltage-frequency conversion circuit, and in particular to an RC oscillation circuit.

### BACKGROUND ART

Conventionally, conversion to an analog value and a digital value (A/D conversion) is used to measure an analog amount such as voltage, current, capacitance, and the like. There are various methods including an integration type, a successive approximation type, and a ΔΣ type, where a conversion method most suited for the target analog amount is selected. An IC integrating such circuits has been commercialized from various manufacturers.

However, the cost of such IC is high and the control by software is necessary.

Furthermore, if the resolution is enhanced to carry out a highly accurate measurement, the cost tends to become higher by such amount.

In practical use, frequency enables the most reliable and highly accurate measurement, and thus, ND conversion of low cost and high accuracy can be enabled using the frequency.

For instance, Japanese Unexamined Patent Publication No. 9-113310 discloses a piezo resistance type sensor device, and also discloses a method of correcting the variation of the sensor and converting the same to frequency.

Japanese Unexamined Patent Publication No. 10-104292 discloses a capacitance type sensor device, and also discloses a circuit for converting a capacitance component that changes according to a pressure to frequency.
Patent Document 1: Japanese Unexamined Patent Publication No. 9-113310
Patent Document 2: Japanese Unexamined Patent Publication No. 10-104292

### SUMMARY OF INVENTION

However, the piezo resistance type sensor device described in Japanese Unexamined Patent Publication No. 9-113310 discloses a method using a CR oscillation circuit but adopts a complex conversion method of calculating a periodical time difference of the oscillating frequencies oscillated from the two CR oscillation circuits, and hence, the sensor device is expensive. The capacitance type sensor device described in Japanese Unexamined Patent Publication No. 10-104292 is easily subjected to the influence of temperature characteristics and is expensive.

Therefore, one or more embodiments of the present invention provides a voltage-frequency conversion circuit having high accuracy with a simple method and a blood pressure measurement device equipped with the same.

According to one or more embodiments of the present invention, a voltage-frequency conversion circuit includes an RC oscillation circuit with a capacitance component and a resistance component. The RC oscillation circuit includes an input terminal to which an input voltage is input, a first resistor element connected between the input terminal and a first internal node, a first capacitor having one electrode connected to the first internal node and the other electrode connected to a second internal node, a second resistor element having one conductive terminal connected to the first internal node in parallel with the first capacitor, a first logic circuit connected to the other conductive terminal of the second resistor element and connected between the first internal node and the second internal node through the second resistor element, a second logic circuit, connected to the second internal node, for outputting an oscillation signal in accordance with an output signal of the first logic circuit, and a first switch element for electrically connecting the first internal node connected to one electrode to a fixed voltage to charge and discharge the first capacitor according to a voltage level of the second internal node.

According to one or more embodiments of the present invention, input voltage corresponds to the output voltage of the piezo resistance type sensor.

According to one or more embodiments of the present invention, the first switch element is conducted when the voltage level of the second internal node is greater than or equal to the threshold value, and the first internal node connected to one electrode is electrically connected to the fixed voltage so that the first capacitor is discharged. The first switch element is non-conducted when the voltage level of the second internal node is smaller than the threshold value, and the first internal node connected to one electrode is connected to the input voltage so that the first capacitor is charged.

There are arranged a third resistor element connected between the input terminal and a third internal node, a second capacitor having one electrode connected to the third internal node and the other electrode connected to a fourth internal node, and a fourth resistor element having one conductive terminal connected to the third internal node in parallel with the second capacitor. The first logic circuit includes a first inverter circuit connected to the other conductive terminal of the second resistor element, and an exclusive OR circuit for receiving the input of the output terminal of the third inverter circuit and the other conductive terminal of the fourth resistor element and outputting the input to the second internal node. The second logic circuit includes a second inverter connected between the second internal node and the fourth internal node, and a third inverter circuit connected to the fourth internal node. There is further arranged a second switch element for electrically connecting the third internal node connected to the one electrode and the fixed voltage to discharge the second capacitor according to the voltage level of the fourth internal node.

According to one or more embodiments of the present invention, a blood pressure measurement device includes a cuff to be wrapped around a predetermined measurement site of a person to be measured, and pressure detection means for detecting the pressure of the cuff. The pressure detection means includes a piezo resistance type sensor for generating voltage in accordance with the pressure of the cuff, and an RC oscillation circuit including a capacitance component and a resistance component. The RC oscillation circuit includes an input terminal to which an input voltage is input, a first resistor element connected between the input terminal and a first internal node, a first capacitor having one electrode connected to the first internal node and the other electrode connected to a second internal node, a second resistor element having one conductive terminal connected to the first internal node in parallel with the first capacitor, a first logic circuit connected to the other conductive terminal of the second resistor element and connected between the first internal node and the second internal node through the second resistor element, a second logic circuit, connected to the second internal node, for outputting an oscillation signal in accordance with an output signal of the first logic circuit, and a first switch element for electrically connecting the first internal node connected to one electrode to a fixed voltage to charge and discharge the first capacitor according to a voltage level of the second internal node.

The voltage-frequency conversion circuit and the blood pressure measurement device according to one or more embodiments of the present invention have the first switch element charging or discharging the first capacitor according to the output signal of the first logic circuit. The charging time of the first capacitor changes according to the input voltage input to the input terminal, and hence, the frequency of the oscillation signal can be adjusted with a simple method.

### BRIEF DESCRIPTION OF THE DRAWINGS

FIG. 1 is a perspective view of an outer appearance of a sphygmomanometer 1 according to one or more embodiments of the present invention.

FIG. 2 is a block diagram showing a hardware configuration of the sphygmomanometer 1 according to one or more embodiments of the present invention.

FIG. 3 is a view describing a piezo resistance type pressure sensor 32 according to one or more embodiments of the present invention.

FIG. 4 is a view describing a conventional RC oscillation circuit.

FIG. 5 is a view describing a voltage level of each node of the conventional RC oscillation circuit.

FIG. 6 is a view describing a voltage-frequency conversion circuit according to one or more embodiments of the present invention.

FIG. 7 is a view describing the voltage level of each node of the voltage-frequency conversion circuit according to one or more embodiments of the present invention.

FIG. 8 is a view describing a voltage-frequency conversion circuit according to one or more embodiments of the present invention.

FIG. 9 is a view describing the voltage level of each node of the voltage-frequency conversion circuit according to one or more embodiments of the present invention.

### DETAILED DESCRIPTION OF INVENTION

Embodiments of the present invention will be described in detail with reference to the drawings. The same reference numerals are denoted for the same or corresponding portions in the figures, and the description thereof will not be repeated.

<Regarding Outer Appearance and Configuration>

First, the outer appearance and the configuration of a blood pressure measurement device (hereinafter referred to as “sphygmomanometer”) 1 according to one or more embodiments of the present invention will be described.

(Regarding Outer Appearance)

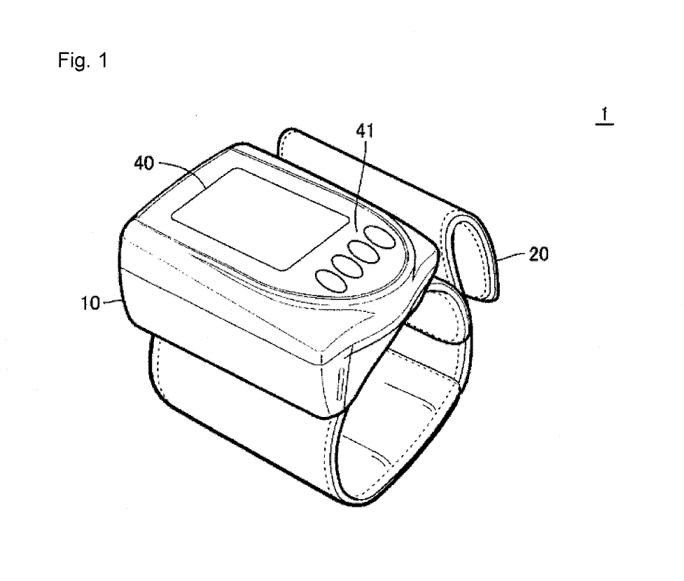

The sphygmomanometer 1 according to one or more embodiments of the present invention will be described using FIG. 1.

With reference to FIG. 1, the sphygmomanometer 1 includes a main body 10 and a cuff 20 that can be wrapped around the wrist of a person to be measured. The main body 10 is attached to the cuff 20. A display unit 40 configured by liquid crystal or the like, and an operation unit 41 for accepting instruction from the user (representatively person to be measured) are arranged on the surface of the main body 10. The operation unit 41 includes a plurality of switches.

(Regarding Hardware Configuration)

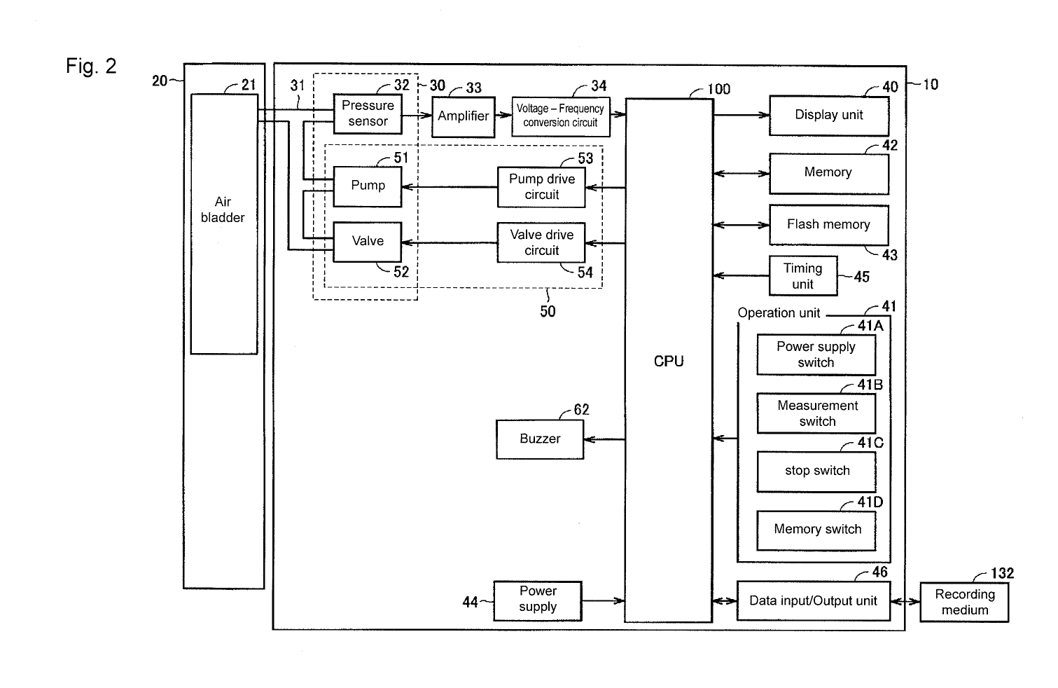

The hardware configuration of the sphygmomanometer 1 according to one or more embodiments of the present invention will be described using FIG. 2.

With reference to FIG. 2, the cuff 20 of the sphygmomanometer 1 includes an air bladder 21. The air bladder 21 is connected to an air system 30 through an air tube 31.

In addition to the display unit 40 and the operation unit 41, the main body 10 includes the air system 30, a CPU (Central Processing Unit) 100 for controlling each unit in a concentrated manner and performing various types of calculation processes, a memory 42 for storing programs for causing the CPU 100 to carry out a predetermined operation and various types of data, a nonvolatile memory (e.g., flash memory) 43 for storing the measured blood pressure, a power supply 44 for supplying power to the CPU 100 or the like, a clock unit 45 for carrying out the timing operation, a data input/output unit 46 for accepting input of data from the outside, and a buzzer 62 for issuing a warning sound or the like.

The operation unit 41 includes a power supply switch 41A for accepting input of the instruction to turn ON or OFF the power supply, a measurement switch 41B for accepting instruction to start the measurement, a stop switch 41C for accepting the instruction to stop the measurement, and a memory switch 41D for accepting the instruction to read out information such as blood pressure recorded in the flash memory 43. The operation unit 41 may also include an ID switch (not shown) operated to input the ID (Identification) information for identifying the person to be measured. The recording and reading of the measurement data thus can be carried out for each person to be measured.

The air system 30 includes a pressure sensor 32 for detecting the pressure (cuff pressure) of the air bladder 21, a pump 51 for supplying air to the air bladder 21 to pressurize the cuff pressure, and a valve 52 to be opened and closed to exhaust or enclose the air of the air bladder 21.

The main body 10 further includes an amplifier 33, a voltage-frequency conversion circuit (oscillation circuit) 34, a pump drive circuit 53, and a valve drive circuit 54 in relation to the air system 30.

In the present example, the pressure sensor 32 is a piezo resistance type pressure sensor by way of example. The amplifier 33 amplifies the output voltage of the pressure sensor 32 and outputs the same to the voltage-frequency conversion circuit 34. The voltage-frequency conversion circuit 34 outputs a signal having an oscillating frequency in accordance with the output voltage of the pressure sensor 32 through the amplifier 33 to the CPU 100. The voltage-frequency conversion circuit 34 will be described later. The amplifier 33 is arranged to amplify the difference because the voltage level difference (amplitude) of the output signal from the pressure sensor 32 is small, but does not need to be particularly arranged if the voltage level difference (amplitude) of the output signal from the pressure sensor 32 is large and a configuration of directly connecting to the pressure sensor 32 may be adopted.

The CPU 100 converts the oscillating frequency obtained from the voltage-frequency conversion circuit 34 to a pressure, and detects the pressure. The pump drive circuit 53 controls the drive of the pump 51 based on a control signal provided from the CPU 100. The valve drive circuit 54 performs the open/close control of the valve 52 based on a control signal provided from the CPU 100.

The pump 51, the valve 52, the pump drive circuit 53 and the valve drive circuit 54 configure an adjustment mechanism 50 for adjusting the cuff pressure. The device for adjusting the cuff pressure is not limited thereto.

For example, the data input/output unit 46 performs reading and writing programs and data from and to the removable recording medium 132. The data input/output unit 46 may execute transmission and reception of programs and data through a communication line from an external computer (not shown).

As shown in FIG. 1, the sphygmomanometer 1 according to one or more embodiments of the present invention has the main body 10 attached to the cuff 20, but the main body 10 and the cuff 20 may be connected by an air tube (air tube 31 in FIG. 2) as adopted in the upper arm sphygmomanometer.

The air bladder 21 is arranged in the cuff 20, but the fluid supplied to the cuff 20 is not limited to air, and may be liquid or gel. Alternatively, it is not limited to fluid, and may be uniform fine particles such as micro-beads.

According to one or more embodiments of the present invention, a predetermined measurement site is the wrist, but is not limited thereto, and may be other sites such as the upper arm.

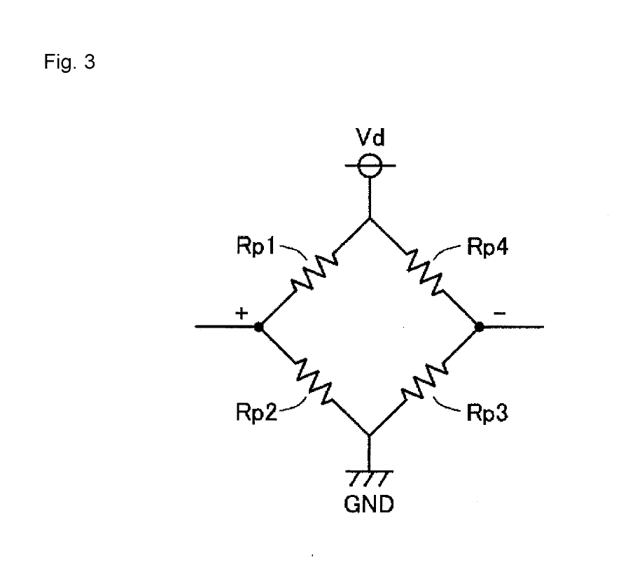

The piezo resistance type pressure sensor 32 according to one or more embodiments of the present invention will be described using FIG. 3.

With reference to FIG. 3, the pressure sensor 32 includes resistor elements Rp1 to Rp4 connected in parallel between a power supply voltage Vd and a ground voltage GND, which is the fixed voltage. A connection node between the resistor elements Rp1 and Rp2 is connected to an output terminal (+) side. A connection node between the resistor elements Rp3 and Rp4 is connected to an output terminal (−) side. The piezo resistance type pressure sensor produces a potential difference at the output terminal as the resistance value of each resistor element changes according to pressure. The pressure sensor 32 outputs the voltage signal generated at the output terminal to the voltage-frequency conversion circuit 34 through the amplifier 33.

The conventional RC oscillation circuit will be described first.

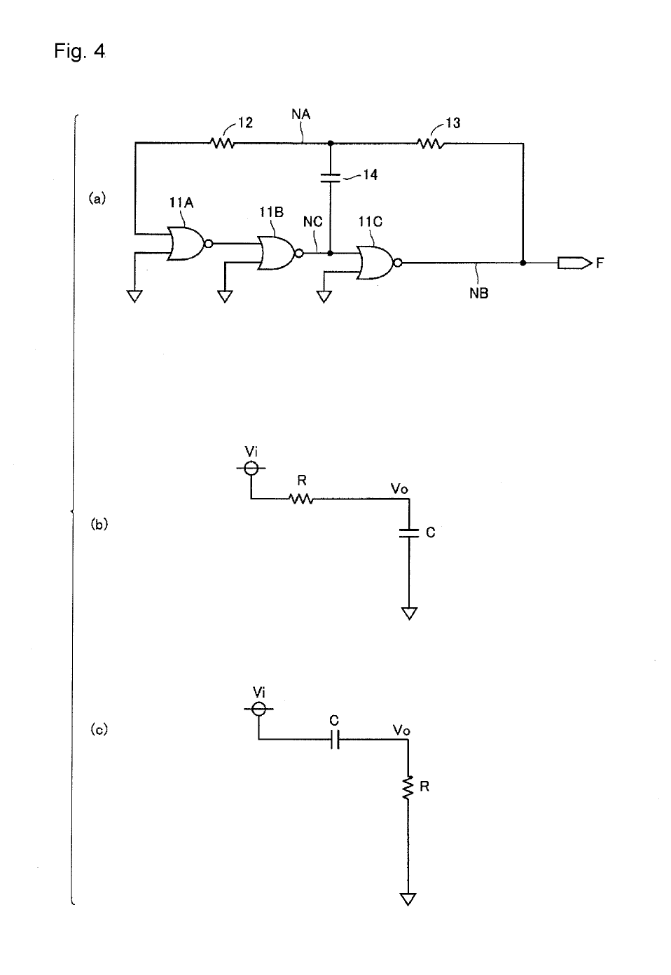

The conventional RC oscillation circuit will be described using FIG. 4.

With reference to FIG. 4(a), the conventional RC oscillation circuit includes resistor elements 12, 13, NOR circuits 11A to 11C and a capacitor 14.

The resistor element 13 is arranged between the node NA and the node NB. The resistor element 12 is arranged between the node NA and one side of the input node of the NOR circuit 11A.

The capacitor 14 has one electrode connected to the node NA and the other electrode connected to the node NC. One side of the input node of the NOR circuit 11A is connected to the node NA through the resistor element 12 and the other side is connected to the ground voltage GND that is the fixed voltage, and the exclusive NOR logical calculation result is output to one side of the input node of the NOR circuit 11B.

One side of the input node of the NOR circuit 11B is connected to the output node of the NOR circuit 11A and the other side of the input node of the NOR circuit 11B is connected to the ground voltage GND that is the fixed voltage, and the exclusive NOR logical calculation result is transmitted to the node NC of the NOR circuit 11C.

One side of the input node of the NOR circuit 11C is connected to the node NC and the other side is connected to the ground voltage GND that is the fixed voltage, and the exclusive NOR logical calculation result is transmitted to the output node NB.

The other input node of the NOR circuit 11A, 11B, 11C is connected to the ground voltage GND. Therefore, the NOR circuit 11A, 11B, 11C each functions as an inverter circuit for inverting and outputting the input signal.

The operation of the RC oscillation circuit will now be described.

The RC oscillation circuit has the oscillating frequency set by the time until reaching the threshold value of the NOR circuit 11A by the time constant circuit including the resistor element 13 and the capacitor 14.

Specifically, if the input node of the NOR circuit 11A is set to “L” level and the output of the NOR circuit 11A becomes “H” level, the node NB is also set to “H” level through the NOR circuits 11B, 11C.

If the capacitor 14 is charged and the voltage of the node NA becomes “H” level, one input node of the NOR circuit 11A also becomes “H” level and the output level of the NOR circuit 11A changes. Accompanied therewith, the output level of the NOR circuit 11A is set from “H” level to “L” level, so that the node NB is also set to “L” level through the NOR circuits 11B, 11C.

In turn, when the electric charges accumulated in the capacitor 14 are discharged and the voltage level of the node NA becomes “L” level, one input node of the NOR circuit 11A also becomes “L” level, so that the output level of the NOR circuit 11A changes from “L” level to “H” level. The node NB is also set to “H” level through the NOR circuits 11B, 11C.

The charging operation and the discharging operation are repeated, so that the voltage of the node NB is alternately output at “L” level and “H” level to become an oscillating operation.

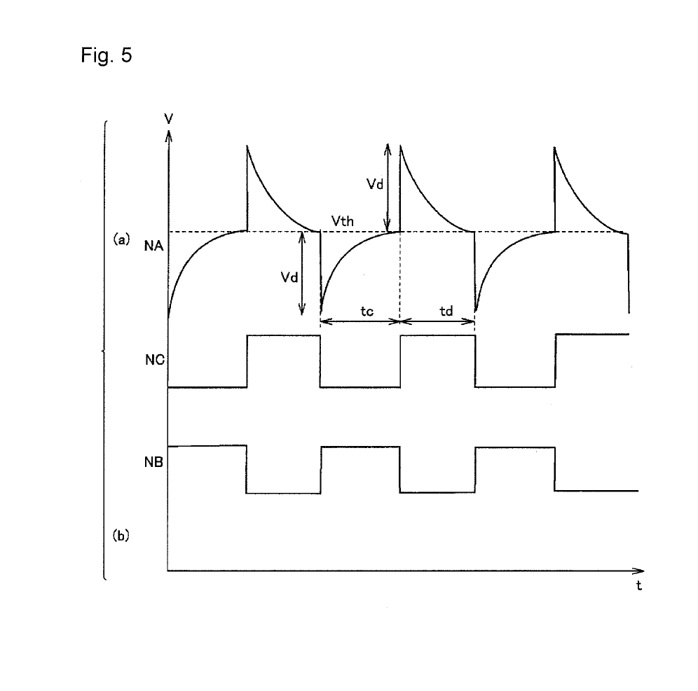

The voltage level of each node of the conventional RC oscillation circuit will now be described using FIG. 5.

With reference to FIG. 5, the voltage waveforms of the nodes NA, NB, NC are shown.

The period of the charging operation and the discharging operation will be described.

FIG. 4(b) is a view describing the charging operation of a typical time constant circuit configured by a resistance value R and a capacitance C.

In other words, the resistance value R corresponds to the resistance component of the resistor element 13 of FIG. 4(a), and the capacitance C corresponds to the capacitance component of the capacitor 14 of FIG. 4(a).

The voltage Vo of the time constant circuit is expressed with the following equation.

$$\begin{matrix}[ Equation 1 ] & \\ \frac{Vi - Vo}{R} - C \frac{d Vo}{d t} \\ \frac{1}{RC} \int d t = \int \frac{1}{Vi - Vo} d Vo \\ \frac{t}{RC} + A = - log ( Vi - Vo ) A \text{:} integral constant \\ Vi - Vo = {e}^{- \frac{t}{RC} + A} \\ Vo = Vi - B {e}^{- \frac{t}{RC}} & ( 1 )\end{matrix}$$

The initial condition for calculating the integral constant A is such that the voltage Vo is expressed with the following equation if voltage Vo=0 when time t=0.

$$\begin{matrix}[ Equation 2 ] & \\ 0 = Vi - B \\ B = Vi \\ \begin{matrix}∴ Vo = Vi - Vi {e}^{- \frac{t}{RC}} \\ = Vi ( 1 - {e}^{- \frac{t}{RC}} ) .\end{matrix} & ( 2 )\end{matrix}$$

On the other hand, the initial condition of the charging operation of the RC oscillation circuit shown in FIG. 4(a) is that the charging operation starts immediately after the voltage reaches Vth by the discharging operation. In other words, when time t=0, the voltage Vo of the node Na becomes Vth-Vd.

Therefore, when the initial condition is substituted into Equation (1), the following equation is obtained.

$$\begin{matrix}[ Equation 3 ] & \\ Vth - Vd = Vd - B ( Vi = Vd ) \\ B = 2 Vd - Vth \\ Vo = Vd - ( 2 Vd - Vth ) {e}^{- \frac{t}{RC}} & ( 3 )\end{matrix}$$

Solving the above for t, the following equation is obtained.

$$\begin{matrix}[ Equation 4 ] & \\ ( 2 Vd - Vth ) {e}^{- \frac{t}{RC}} = Vd - Vo \\ {e}^{- \frac{t}{RC}} = \frac{Vd - Vo}{2 Vd - Vth} \\ t = - RC log ( \frac{Vd - Vo}{2 Vd - Vth} ) & ( 4 )\end{matrix}$$

When the voltage Vo is transmitted to the input node of the NOR circuit 11A and reaches the threshold value Vth of the NOR circuit 11A, the output level of the NOR circuit 11A changes and is set to “L” level. In other words, the time to reach the threshold value Vth of the NOR gate is the time when Vo=Vth. The threshold value Vth of the NOR gate is expressed with the following equation by substituting into the above equation because the threshold value Vth is generally ½ of the power supply voltage Vd.

$$\begin{matrix}[ Equation 5 ] & \\ \begin{matrix}Vo = Vd - ( 2 Vd - \frac{1}{2} Vd ) {e}^{- \frac{t}{RC}} \\ = Vd - \frac{3}{2} Vd {e}^{- \frac{t}{RC}} \\ = Vd - ( 1 - \frac{3}{2} {e}^{- \frac{t}{RC}} )\end{matrix} & ( 5 )\end{matrix}$$

The time tc required for the charging operation is expressed with the following equation.

$$\begin{matrix}[ Equation 6 ] & \\ \begin{matrix}t = - RC log ( \frac{Vd - \frac{1}{2} Vd}{2 Vd - \frac{1}{2} Vd} ) \\ = - RC log \frac{\frac{1}{2}}{\frac{3}{2}} Vd \\ tc = - RC log \frac{1}{3} Vd\end{matrix} & ( 6 )\end{matrix}$$

The discharging operation will now be considered.

FIG. 4(c) is a view describing the discharging operation of a typical time constant circuit configured by a resistance value R and a capacitance C.

In other words, the resistance value R corresponds to the resistance component of the resistor element 13 of FIG. 4(a), and the capacitance C corresponds to the capacitance component of the capacitor 14 of FIG. 4(a).

The voltage Vo of the time constant circuit is expressed with the following equation.

$$\begin{matrix}[ Equation 7 ] & \\ C \frac{d ( Vi - Vo )}{d t} = \frac{Vo}{R} \\ - \frac{d Vo}{d t} = \frac{Vo}{RC} \\ \int \frac{1}{Vo} d Vo = - \frac{1}{RC} \int d t \\ log Vo = - \frac{t}{RC} + A \\ Vo = B {e}^{- \frac{t}{RC}} & ( 7 )\end{matrix}$$

The initial condition of the discharging operation of the RC oscillation circuit shown in FIG. 4(a) is that the discharging operation starts immediately after the voltage reaches Vth by the charging operation. In other words, when time t=0, the voltage Vo of the node NA becomes Vth+Vd.

Therefore, when the initial condition is substituted into Equation (7), the following equation is obtained.

$$\begin{matrix}[ Equation 8 ] & \\ Vth + Vd = B \\ ∴ Vo = ( Vth + Vd ) {e}^{- \frac{t}{RC}} & ( 8 )\end{matrix}$$

Solving the above for t, the following equation is obtained.

$$\begin{matrix}[ Equation 9 ] & \\ t = - RC log ( \frac{Vo}{Vth + Vd} ) & ( 9 )\end{matrix}$$

The time to reach the threshold value Vth of the NOR gate is the time when Vo=Vth. The threshold value Vth of the NOR gate is expressed with the following equation by substituting into the above equation because the threshold value Vth is generally ½ of the power supply voltage Vd.

$$\begin{matrix}[ Equation 10 ] & \\ Vo = \frac{3}{2} Vd {e}^{- \frac{t}{RC}} & ( 10 )\end{matrix}$$

The time td required for the discharging operation is expressed with the following equation.

$$\begin{matrix}[ Equation 11 ] & \\ td = - RC log \frac{1}{3} Vd & ( 11 )\end{matrix}$$

Therefore, the RC oscillation circuit shown in FIG. 4(a) can obtain the pulse waveform of duty 50% because the RC oscillation circuit has a relationship of time tc=td.

As described above, the total time of the time tc required for the charging operation and the time td required for the discharging operation becomes one period.

Therefore, the oscillating frequency can be changed by changing the resistance component, the capacitance component, or the like, as apparent from Equations (6) and (11).

In the conventional capacitance type sensor device, the RC oscillating circuit is used, and a method of changing the oscillating frequency by changing the capacitor capacity is adopted.

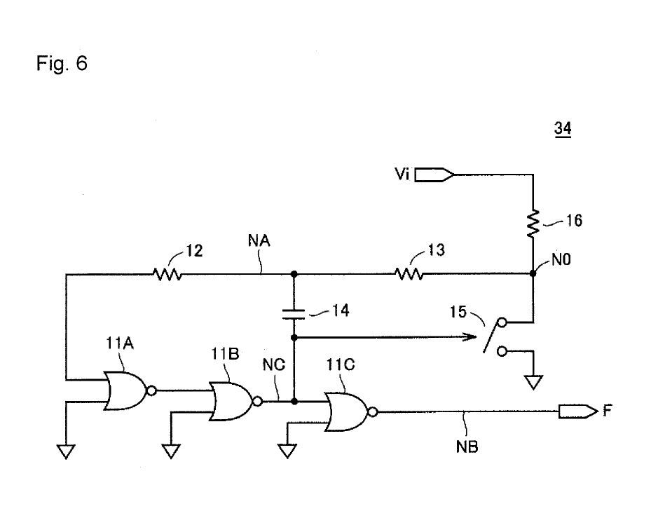

The voltage-frequency conversion circuit 34 according to one or more embodiments of the present invention will be described using FIG. 6.

With reference to FIG. 6, the voltage-frequency conversion circuit 34 according to one or more embodiments of the present invention includes resistor elements 12, 13, 16, NOR circuits 11A to 11C, a capacitor 14, and a switch element 15.

The resistor element 16 is arranged between the input terminal and the node N0. The switch element 15 is arranged between the node N0 and the ground voltage GND that is a fixed voltage, and is conducted according to the voltage level of the node NC. The resistor element 13 is arranged between the node N0 and the node NA. The resistor element 12 is arranged between the node NA and one side of the input node of the NOR circuit 11A.

The capacitor 14 has one electrode connected to the node NA and the other electrode connected to the node NC. One side of the input node of the NOR circuit 11A is connected to the node NA through the resistor element 12 and the other side is connected to the ground voltage GND that is the fixed voltage, and the exclusive NOR logical calculation result is output to one side of the input node of the NOR circuit 11B.

One side of the input node of the NOR circuit 11B is connected to the output node of the NOR circuit 11A and the other side of the input node of the NOR circuit 11B is connected to the ground voltage GND that is the fixed voltage, and the exclusive NOR logical calculation result is transmitted to the node NC of the NOR circuit 11C.

One side of the input node of the NOR circuit 11C is connected to the node NC and the other side is connected to the ground voltage GND that is the fixed voltage, and the exclusive NOR logical calculation result is transmitted to the output node NB.

Similar to the RC oscillation circuit, in the present example as well, the oscillating frequency is set by the time until reaching the threshold value of the NOR circuit 11A by the time constant circuit including the resistor elements 13, 16 and the capacitor 14.

Specifically, if the input node of the NOR circuit 11A is “L” level, the output signal thereof is set to “H” level. Accompanied therewith, the output signal of the NOR circuit 11B is set to “L” level and the output signal of the NOR circuit 11C is set to “H” level.

Because the voltage level of the node NC is “L” level, one electrode of the capacitor 14 is connected to the input terminal through the resistor elements 13, 16, where the voltage of the node NA is expressed with the following equation by the charging operation by means of the time constant circuit configured by the resistor elements 13, 16 and the capacitor 14. In other words, as described above, the initial condition of the charging operation of the RC oscillation circuit is input using Equation (1).

The initial condition is such that the voltage Vo of the node Na is Vth−Vd when time t=0.

$$\begin{matrix}[ Equation 12 ] & \\ Vo = Vi - B {e}^{- \frac{t}{RC}} \\ t = 0 Vo = Vth - Vd \\ ∴ Vth - Vd = Vi - B \\ B = Vi + Vd - Vth & ( 1 ) \\ Vo = Vi - ( Vi + Vd - Vth ) {e}^{- \frac{t}{RC}} & ( 12 )\end{matrix}$$

Solving the above for t, the following equation is obtained.

$$\begin{matrix}[ Equation 13 ] & \\ t = - RC log ( \frac{Vi - Vo}{Vi + Vd - Vth} ) & ( 13 )\end{matrix}$$

The time to reach the threshold value Vth of the NOR gate is the time when Vo=Vth. The threshold value Vth of the NOR gate is expressed with the following equation by substituting into the above equation because the threshold value Vth is generally ½ of the power supply voltage Vd.

$$\begin{matrix}[ Equation 14 ] & \\ Vo = Vi - ( Vi + \frac{1}{2} Vd ) {e}^{- \frac{t}{RC}} & ( 14 )\end{matrix}$$

The time to required for the charging operation is expressed with the following equation.

$$\begin{matrix}[ Equation 15 ] & \\ te = - RC log \frac{Vi - \frac{1}{2} Vd}{Vi + \frac{1}{2} Vd} & ( 15 )\end{matrix}$$

When the charging voltage is transmitted to the input node of the NOR circuit 11A and reaches the threshold value Vth of the NOR circuit 11A, the output level of the NOR circuit 11A changes and is set to “L” level. Accompanied therewith, the output signal of the NOR circuit 11B is set from “L” level to “H” level. The output signal of the NOR circuit 11C is set from “H” level to “L” level.

The switch element 15 is conducted (ON) according to the voltage level (“H” level) of the node NC with the setting of the output signal of the NOR circuit 11B to “H” level. The ground voltage GND that is the fixed voltage and the node N0 are thereby electrically connected. Accompanied therewith, the voltage of the node NB is expressed with the following equation by the discharge operation by the time constant circuit including the resistor element 13 and the capacitor 14.

$$\begin{matrix}[ Equation 16 ] & \\ td = - RC log ( \frac{1}{3} Vd ) & ( 16 )\end{matrix}$$

In other words, Equation (16) is the same as Equation (11).

When the relevant charging voltage is transmitted to the input node of the NOR circuit 11A and becomes smaller than the threshold value Vth of the NOR circuit 11A, the output level of the NOR circuit 11A changes and is set from “L” level to “H” level.

The output signal of the NOR circuit 11B is set from “H” level to “L” level. The output signal of the NOR circuit 11C is set from “L” level to “H” level.

The switch element 15 is non-conducted (OFF) according to the voltage level (“L” level) of the node NC with the setting of the output signal of the NOR circuit 11B to “L” level. The ground voltage GND that is the fixed voltage and the node N0 are thereby electrically separated. Accompanied therewith, one electrode of the capacitor 14 is connected to the input terminal through the resistor element 13, 16, and thus, the charging operation described above is executed.

In other words, the output signal of the NOR circuit 11C outputs the oscillation signal of “L” level, “H” level, “L” level, . . . according to the charging operation and the discharging operation.

In the voltage-frequency conversion circuit 34 according to one or more embodiments of the present invention, the capacitance component and the resistance component of the capacitor 14 and the resistor elements 12, 13, 16 are fixed values, and the input voltage input to the input terminal changes. The input voltage input to the input terminal is the output voltage output according to the pressure from the pressure sensor.

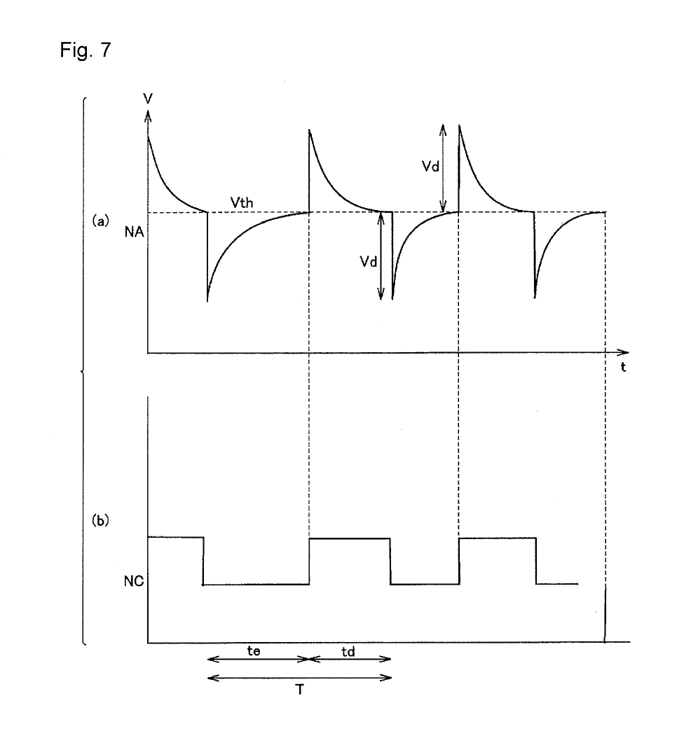

The voltage level of each node of the voltage-frequency conversion circuit 34 according to one or more embodiments of the present invention will be described using FIG. 7.

With reference to FIG. 7, the voltage levels of the node NA and the node NC are shown.

The charging time to changes as shown in Equation (15) in the configuration according to one or more embodiments of the present invention, that is, the configuration in which the input voltage input from the input terminal changes. The discharging time does not change because the capacitance component and the resistance component of the capacitor 14 and the resistor elements 12, 13 16 are fixed. The resistance value R in Equation (15) corresponds to the total value of the resistance components of the resistor elements 13, 16 of FIG. 6. The capacitance C corresponds to the capacitance component of the capacitor 14 of FIG. 6.

As the charging time until reaching the threshold value of the NOR circuit 11A depends on the input voltage, the cycle of the oscillation signal changes and the oscillating frequency can be changed.

In other words, the signal having the oscillating frequency in accordance with the output voltage of the pressure sensor 32 is output to the CPU 100 by the voltage-frequency conversion circuit 34 according to one or more embodiments of the present invention, and the CPU 100 converts the oscillating frequency to pressure and detects the pressure.

Therefore, an inexpensive and highly accurate voltage-frequency conversion circuit can be realized with a simple method. A blood pressure measurement device using the same can also be realized.

In the configuration of FIG. 6, a configuration of the NOR circuit in which one input node is connected to the ground voltage GND (“L” level) that is the fixed voltage has been described, but a configuration in which the NAND circuit is used in place of the NOR circuit may be adopted by connecting one input node to the power supply voltage Vd (“H” level).

In the configuration of FIG. 6, the configuration using the NOR circuits 11A to 11C has been described, where the respective input node is connected to the ground voltage GND that is the fixed voltage and thus functions as an inverter circuit for inverting the logic level of the input signal. Therefore, the configuration in which the NOR circuits 11A to 11C are replaced with the inverter circuits may be adopted. According to such configuration, the number of configuring elements of the circuit can be reduced and the layout of the circuit can be made smaller.

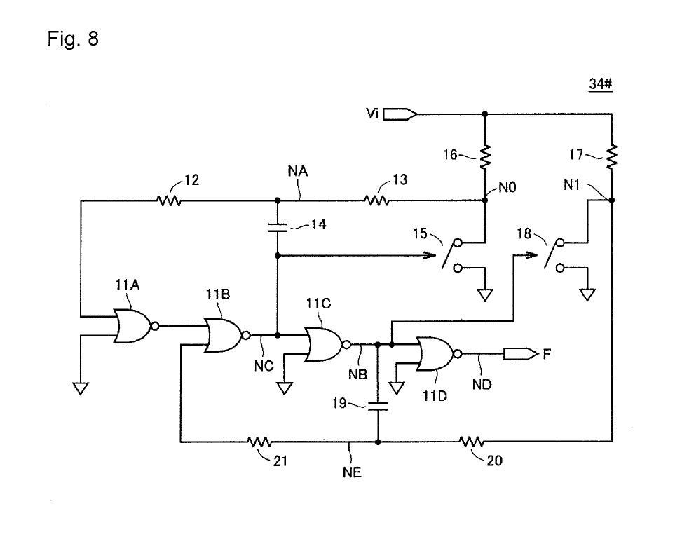

The voltage-frequency conversion circuit 34# according to one or more embodiments of the present invention will be described using FIG. 8.

With reference to FIG. 8, the voltage-frequency conversion circuit 34# according to one or more embodiments of the present invention differs from the voltage-frequency conversion circuit 34 described in FIG. 6 in that an NOR circuit 11D, resistor elements 17, 20, 21, a switch element 18, and a capacitor 19 are further arranged.

Specifically, the resistor element 17 is arranged between the input terminal and the node N1. The switch element 18 is arranged between the node N1 and the fixed voltage, and becomes conductive/non-conductive according to the voltage level of the node NB. The resistor element 20 is arranged between the node NE and the node N1. The capacitor 19 has one electrode connected to the node NE and the other electrode connected to the node NB. The NOR circuit 11D has one input node connected to the node NB and the other one connected to the fixed voltage, and transmits the NOR logical calculation result to the node ND. One conductive terminal of the resistor element 21 is connected to the node NE and the other conductive terminal is connected to the input node of the NOR circuit 11B.

The NOR circuit 11B receives the output signal of the NOR circuit 11A and the signal from the node NE through the resistor element 21, and transmits the exclusive NOR logical calculation result to the node NC.

In the configuration according to one or more embodiments of the present invention as described above, a method in which the charging time is adjusted according to the input voltage so that the period of “H” level of the oscillation signal is adjusted has been described, but a method in which the period of “L” level of the oscillation signal is further adjusted will be described in another configuration according to one or more embodiments of the present invention.

Specifically, if the input node of the NOR circuit 11A is “L” level, the output signal of the NOR circuit 11C is set to “H” level. Accompanied therewith, the output signal of the NOR circuit 11B is set to “L” level and the output signal of the NOR circuit 11C is set to “H” level. The output signal of the NOR circuit 11D is also set to “L” level.

In this case, the node NC is “L” level, and thus, the switch element 15 is non-conductive. The node NB is “H” level, and thus, the switch element 18 is conductive. Therefore, the ground voltage GND that is the fixed voltage and the node N1 are electrically coupled. In other words, the input node input through the resistor elements 20, 21 of the NOR circuit 11B is set to “L” level. Therefore, the NOR circuit 11B functions as the inverter circuit because one input node is “L” level.

Because the voltage level of the node NC is “L” level, one electrode of the capacitor 14 is connected to the input terminal through the resistor elements 13, 16 and the charging operation is executed as described above. When the voltage of the node NA is transmitted to the input node of the NOR circuit 11A by the charging operation and reaches the threshold value Vth of the NOR circuit 11A, the output level of the NOR circuit 11A changes and is set to “L” level. Accompanied therewith, the output signal of the NOR circuit 11B is set from “L” level to “H” level. The output signal of the NOR circuit 11C is set from “H” level to “L” level. The output signal of the NOR circuit 11D is set from “L” level to “H” level.

The switch element 15 is conducted (ON) according to the voltage level (“H” level) of the node NC with the setting of the output signal of the NOR circuit 11B to “H” level. The ground voltage GND that is the fixed voltage and the node N0 are thereby electrically connected. Accompanied therewith, the discharging operation is executed. In this case, the switch element 18 is non-conducted (OFF) because the output signal of the NOR circuit 11C is set from “H” level to “L” level. The NOR circuit 11B functions as the inverter circuit because one input node is “L” level because the output signal of the NOR circuit 11A is “L” level.

The output signal of the NOR circuit 11C is “L” level and the voltage level of the node NB is “L” level, so that one electrode of the capacitor 19 is connected to the input terminal through the resistor elements 17, 20 and the charging operation is executed. When the voltage of the node NE is transmitted to the input node of the NOR circuit 11B by the charging operation and reaches the threshold value Vth of the NOR circuit 11B, the output level of the NOR circuit 11B changes and is set to “L” level. The switch element 15 is then non-conductive (OFF). Therefore, the ground voltage GND that is the fixed voltage and the node N0 are electrically separated. Accompanied therewith, one electrode of the capacitor 14 is connected to the input terminal through the resistor elements 13, 16, and hence, the charging operation described above is executed.

The output level of the NOR circuit 11C is set from “L” level to “H” level with the setting of the output level of the NOR circuit 11B to “L” level. Therefore, the output signal of the NOR circuit 11C is “H” level, and the switch element 18 is conducted. Accompanied therewith, the node N1 is connected to the ground voltage GND. The discharging operation is executed therewith.

In other words, the output signal of the NOR circuit 11D outputs the oscillation signal of “H” level, “L” level, “H” level, “L” level . . . according to the charging operation and the discharging operation.

The voltage-frequency conversion circuit 34# according to one or more embodiments of the present invention has the resistance component and the capacitance component set such that the charging time for the node NE to reach the threshold value Vth of the NOR circuit 11B by the time constant circuit configured by the resistor elements 17, 20 and the capacitor 19 becomes shorter than the discharging time for the node NA to become smaller than or equal to the threshold value Vth of the NOR circuit 11A by the time constant circuit configured by the resistor element 13 and the capacitor 14.

In the voltage-frequency conversion circuit 34# according to one or more embodiments of the present invention, the capacitance component and the resistance component of the capacitors 14, 19 and the resistor elements 12, 13, 16, 17, 20, 21 are fixed values, and the input voltage input to the input terminal changes. The input voltage input to the input terminal is the output voltage output according to the pressure in the pressure sensor.

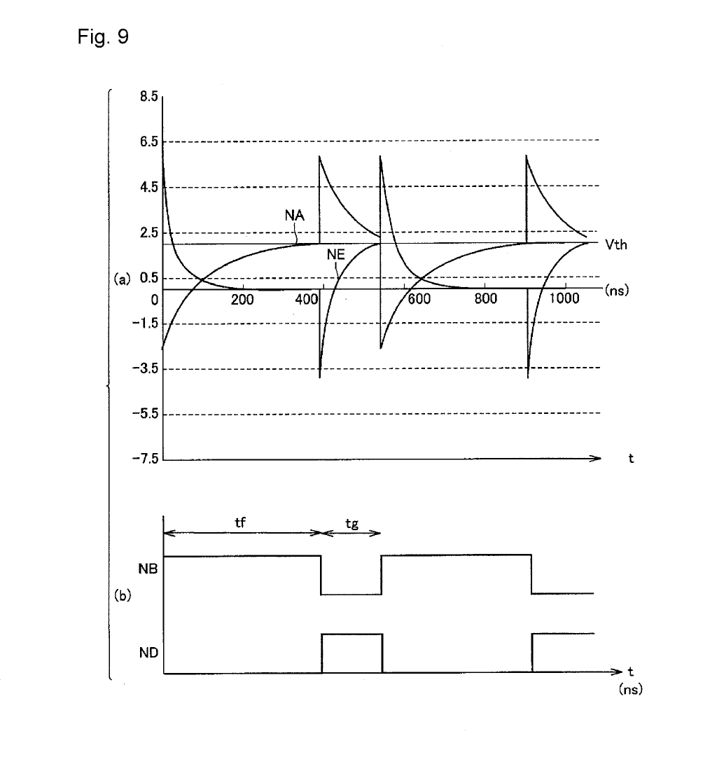

The voltage level of each node of the voltage-frequency conversion circuit 34# according to one or more embodiments of the present invention will be described using FIG. 9.

With reference to FIG. 9(a), the voltage levels of the node NA and the node NE are shown.

The charging time tf of the node NA and the charging time tg of the node NE change in the configuration according to one or more embodiments of the present invention, that is, the configuration in which the input voltage input from the input terminal changes. The discharging time does not change because the capacitance component and the resistance component of the capacitors 14, 19 and the resistor elements 12, 13 16, 17, 20, 21 are fixed.

The charging time of the node NA and the charging time of the node NE will be hereinafter described.

First, the node NE will be described.

For the initial condition at the time of charging, Vo is 0−Vd when t=0.

Therefore, the voltage of the node NE can be expressed as in the following equation by substituting the initial condition.

$$\begin{matrix}[ Equation 17 ] & \\ Vo = Vi - B {e}^{- \frac{t}{RC}} \\ - Vd = Vi - B \\ B = Vi + Vd \\ ∴ Vo = Vi - ( Vi + Vd ) {e}^{- \frac{t}{RC}} & ( 17 )\end{matrix}$$

Solving the above for t, the following equation is obtained.

$$\begin{matrix}[ Equation 18 ] & \\ t = - RC log \frac{Vi - Vo}{Vi + Vd} & ( 18 )\end{matrix}$$

The time of reaching the threshold value Vth of the NOR gate is the time when Vo=Vth.

Therefore, the time tg required for the charging operation is expressed with the following equation.

$$\begin{matrix}[ Equation 19 ] & \\ tg = - RC log \frac{Vi - Vth}{Vi + Vd} & ( 19 )\end{matrix}$$

The resistance value R in Equation (19) corresponds to the total value of the resistance components of the resistor elements 17, 20 of FIG. 8. The capacitance C corresponds to the capacitance component of the capacitor 19 of FIG. 8.

The node NA will now be considered.

First, the initial condition at the time of discharging of the node NA is such that Vo=Vth+Vd when t=0.

Therefore, with respect to the node NA, the node NA at the time of discharging is obtained as Equation (8), as described above.

As described above, the voltage-frequency conversion circuit 34# according to one or more embodiments of the present invention has the resistance component and the capacitance component set such that the charging time for the node NE to reach the threshold value Vth of the NOR circuit 11B by the time constant circuit configured by the resistor elements 17, 20 and the capacitor 19 becomes shorter than the discharging time for the node NA to become smaller than or equal to the threshold value Vth of the NOR circuit 11A by the time constant circuit configured by the resistor element 13 and the capacitor 14.

Therefore, when the node NE reaches the threshold value Vth of the NOR circuit 11B, the node NA is set to a voltage higher than the threshold value Vth by a predetermined voltage as shown in FIG. 9.

The voltage when the node NE reaches the threshold value Vth of the NOR circuit 11B is first obtained.

Specifically, the time tg at which the voltage of the node NE becomes Vth is substituted into Equation (8).

$$\begin{matrix}[ Equation 20 ] & \\ \begin{matrix}Vo = ( Vth + Vd ) {e}^{- \frac{t}{RC} \times ( - RC log \frac{Vi - Vth}{Vi + Vd} )} \\ = ( Vth + Vd ) \cdot {e}^{log \frac{Vi - Vth}{Vi + Vd}}\end{matrix} \\ \frac{Vo}{Vth + Vd} = {e}^{log \frac{Vi - Vth}{Vi + Vd}} \\ log \frac{Vo}{Vth + Vd} = log \frac{Vi - Vth}{Vi + Vd} \\ ∴ \frac{Vo}{Vth + Vd} = \frac{Vi - Vth}{Vi + Vd} \\ Vo = \frac{( Vth + Vd ) ( Vi - Vth )}{Vi + Vd} = K & ( 20 )\end{matrix}$$

The relevant voltage is the voltage of the node NA when the voltage of the node NE becomes Vth.

The initial condition at the time of the charging operation of the node NA is such that Vo=K−Vd when t=0, and hence, the voltage of the node NA is expressed with the following equation when the initial condition is substituted into Equation (1).

$$\begin{matrix}[ Equation 21 ] & \\ k - Vd = Vi - B \\ B = Vi + Vd - K \\ Vo = Vi - ( Vi + Vd - K ) {e}^{- \frac{t}{RC}} & ( 21 )\end{matrix}$$

Solving the above for t, the time tf required for the charging operation is expressed with the following equation.

$$\begin{matrix}[ Equation 22 ] & \\ \begin{matrix}tf = - RC log \frac{Vi - Vth}{Vi + Vd - k} \\ = - RC log \frac{Vi - Vth}{Vi + Vd - \frac{( Vth + Vd ) ( Vi - Vth )}{Vi + Vd}}\end{matrix} & ( 22 )\end{matrix}$$

The resistance value R in Equation (22) corresponds to the total value of the resistance components of the resistor elements 13, 16 of FIG. 8. The capacitance C corresponds to the capacitance component of the capacitor 14 of FIG. 8.

Therefore, because the charging time until reaching the threshold values of the NOR circuit 11A and the NOR circuit 11B depends on the input voltage, the cycle of the oscillation signal changes and the oscillating frequency can be changed.

In other words, the signal having the oscillating frequency in accordance with the output voltage of the pressure sensor 32 is output to the CPU 100 by the voltage-frequency conversion circuit 34# according to one or more embodiments of the present invention, and the CPU 100 converts the oscillating frequency to pressure and detects the pressure.

Therefore, an inexpensive and highly accurate voltage-frequency conversion circuit can be realized with a simple method. The blood pressure measurement device using the same can also be realized.

In the configuration according to one or more embodiments of the present invention, the charging time is adjusted according to the input voltage by the time constant circuit configured by the resistor elements 13, 16 and the capacitor 14 to adjust the period of “H” level of the node NB of the oscillation signal, and furthermore, the charging time is adjusted according to the input voltage by the time constant circuit configured by the resistor elements 17, 20 and the capacitor 19 to adjust the period of “L” level of the node NB of the oscillation signal.

The oscillating frequency serving as the oscillation signal of the NOR circuit 11D for outputting an inverted signal of the node NB is adjusted.

In the configuration of FIG. 8, the configuration of the NOR circuits 11A, 11C, and 11D in which one input node is connected to the ground voltage GND (“L” level) that is the fixed voltage has been described, but a configuration in which the NAND circuit is used in place of the NOR circuit may be adopted by connecting one input node to the power supply voltage Vd (“H” level).

In the configuration of FIG. 8, the configuration in which the inverter circuit for inverting the logic level of the input signal is replaced may be adopted instead of the configuration of the NOR circuits 11A, 11C, and 11D. According to such configuration, the number of configuring elements of the circuit can be reduced and the layout of the circuit can be made smaller.

According to the configuration of one or more embodiments of the present invention, the period of “H” level and the period of “L” level of the oscillation signal are adjusted according to the input voltage so that a wide dynamic range can be obtained, whereby a more accurate voltage-frequency conversion circuit can be realized. The blood pressure measurement device using the same is also realized.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### DESCRIPTION OF REFERENCE NUMERALS

1 sphygmomanometer
10 main body
20 cuff
21 air bladder
30 air system
31 air tube
32 pressure sensor
33 amplifier
34, 34# voltage-frequency conversion circuit
40 display unit
41 operation unit
41A power supply switch
41B measurement switch
41C stop switch
41D memory switch
42 memory
43 flash memory
44 power supply
45 clock unit
46 data input/output unit
50 adjustment mechanism
51 pump
52 valve
53 pump drive circuit
54 valve drive circuit
62 buzzer
100 CPU
132 recording medium

1. A voltage-frequency conversion circuit comprising:
an RC oscillation circuit comprising a capacitance component and a resistance component,
wherein the RC oscillation circuit comprises:
an input terminal to which an input voltage is input;
a first resistor element connected between the input terminal and a first internal node;
a first capacitor comprising one electrode connected to the first internal node and another electrode connected to a second internal node;
a second resistor element comprising one conductive terminal connected to the first internal node in parallel with the first capacitor;
a first logic circuit connected to another conductive terminal of the second resistor element and connected between the first internal node and the second internal node through the second resistor element;
a second logic circuit, connected to the second internal node, that outputs an oscillation signal in accordance with an output signal of the first logic circuit; and
a first switch element that electrically connects the first internal node connected to the one electrode to a fixed voltage to charge and discharge the first capacitor according to a voltage level of the second internal node.

2. The voltage-frequency conversion circuit according to claim 1, wherein the input voltage corresponds to an output voltage of a piezo resistance type sensor.

3. The voltage-frequency conversion circuit according to claim 1,
wherein the first switch element is conducted when a voltage level of the second internal node is greater than or equal to a threshold value, and the first internal node connected to the one electrode is electrically connected to the fixed voltage so that the first capacitor is discharged, and
wherein the first switch element is non-conducted when the voltage level of the second internal node is smaller than the threshold value, and the first internal node connected to the one electrode is connected to the input voltage so that the first capacitor is charged.

4. The voltage-frequency conversion circuit according to claim 1, further comprising:
a third resistor element connected between the input terminal and a third internal node;
a second capacitor comprising one electrode connected to the third internal node and the other electrode connected to a fourth internal node; and
a fourth resistor element comprising one conductive terminal connected to the third internal node in parallel with the second capacitor,
wherein the first logic circuit comprises a first inverter circuit connected to the other conductive terminal of the second resistor element, and an exclusive OR circuit that receives an input of an output terminal of the first inverter circuit and the other conductive terminal of the fourth resistor element and outputs to the second internal node,
wherein the second logic circuit comprises a second inverter connected between the second internal node and the fourth internal node, and a third inverter circuit connected to the fourth internal node, and
wherein a second switch element that electrically connects the third internal node connected to the one electrode to the fixed voltage to discharge the second capacitor according to the voltage level of the fourth internal node is further arranged.

5. A blood pressure measurement device comprising:
a cuff to be wrapped around a predetermined measurement site of a person to be measured; and
pressure detection means for detecting the pressure of the cuff,
wherein the pressure detection means comprises:
a piezo resistance type sensor that generates a voltage in accordance with the pressure of the cuff; and
an RC oscillation circuit comprising a capacitance component and a resistance component,
wherein the RC oscillation circuit comprises:
an input terminal to which an input voltage is input;
a first resistor element connected between the input terminal and a first internal node;
a first capacitor comprising one electrode connected to the first internal node and another electrode connected to a second internal node;
a second resistor element comprising one conductive terminal connected to the first internal node in parallel with the first capacitor;
a first logic circuit connected to another conductive terminal of the second resistor element and connected between the first internal node and the second internal node through the second resistor element;
a second logic circuit, connected to the second internal node, that outputs an oscillation signal in accordance with an output signal of the first logic circuit; and
a first switch element that electrically connects the first internal node connected to the one electrode to a fixed voltage to charge and discharge the first capacitor according to a voltage level of the second internal node.
